(19) United States
(12) Patent Application Publication
(10) Pub. No.: US 2026/0324985 A1
(43) Pub. Date: Oct. 22, 2026

(54) PRESSURE-BASED SIZING OF BODY VESSELS

(71) Applicant: MUFFIN INCORPORATED, West Lafayette, IN (US)

(72) Inventors: Joshua Krieger, Topsfield, MA (US); Neal Fearnot, West Lafayette, IN (US); Sarah Robbins, Lafayette, IN (US); Gabriel Converse, Lafayette, IN (US)

(73) Assignee: MUFFIN INCORPORATED, West Lafayette, IN (US)

(21) Appl. No.: 19/110,526

(22) PCT Filed: Sep. 14, 2023
(86) PCT No.: PCT/US2023/032728

§ 371 Date: Mar. 11, 2025

Related U.S. Application Data
(60) Provisional application No. 63/406,977, filed on Sep. 15, 2022.

(51) Int. Cl. A61B 5/02; A61B 5/00; A61B 5/055

(52) U.S. Cl. CPC A61B 5/02007; A61B 5/055; A61B 5/6853; A61B 5/6876

(57) ABSTRACT

The disclosure relates to interventional medical devices, such as sizing catheters, useful in the sizing of body vessels for various purposes, such as subsequent implantation of intraluminal medical devices. The disclosure also relates to methods of determining a sizing dimension of a body vessel at an area of interest, methods of implanting an intraluminal medical device at an area of interest in a body vessel, and methods of making intraluminal medical devices.

### FIELD

The disclosure relates generally to the field of medical devices. More particularly, the disclosure relates to interventional medical devices, such as catheters, kits, and methods useful in the sizing of body vessels for various purposes, such as subsequent implantation of intraluminal medical devices. The disclosure also relates to methods of implanting an intraluminal medical device at an area of interest in a body vessel and methods of making intraluminal medical devices.

### BACKGROUND

Measurement of vessel dimensions using imaging techniques and equipment, such as venography and intravascular ultrasound, is frequently used to size body vessels prior to implantation of an intraluminal medical device within a body vessel. Vessel sizing can be an important factor in achieving desirable treatment outcomes following implantation of intraluminal medical devices, such as stents, valves, filters and other devices.

Sizing of compliant body vessels, such as veins, lymphatics, and other body vessels, is particularly important to achieving desirable outcomes since these body vessels naturally distend to a range of diameters in response to variations in pressure exerted on the inner surface of the wall of the body vessel. For example, veins distend to a range of diameters as internal pressure varies as fluid flows through the body vessel. The ability of these body vessels to naturally distend elevates the importance of vessel sizing before implantation of an intraluminal medical device. If a body vessel distends beyond the diameter of a device that has been implanted within the body vessel, the device may migrate from the position at which it was originally, and intentionally, implanted.

Unfortunately, the very property that elevates the importance of pre-implantation vessel sizing—distention—also makes the sizing of these body vessels particularly challenging. Conventional sizing devices and techniques rely on devices and techniques that fail to incorporate or otherwise account for vessel distention.

A need remains, therefore, for new and improved interventional medical devices, kits, and methods useful in the sizing of body vessels for various purposes, such as subsequent implantation of intraluminal medical devices. A need for related methods, such as methods of implanting an intraluminal medical device at an area of interest in a body vessel and methods of making intraluminal medical devices, also exists.

### BRIEF SUMMARY OF SELECTED EXAMPLES

Various example interventional medical devices useful in the sizing of body vessels are described.

An example interventional medical device comprises a sizing catheter having an elongate main body having a proximal end and a distal end and a balloon disposed on the main body adjacent the distal end and defining a cavity. The main body defines an inflation lumen having a proximal opening on or near the proximal end of the main body and a distal opening disposed within the cavity of the balloon. The balloon is movable between an inflated configuration and a deflated configuration by passing fluid through the inflation lumen and into the cavity and withdrawing fluid from the cavity and through the inflation lumen. The balloon has an axial length along which the pressure transmitted to tissue contacting the balloon is known for a particular inflation pressure. In some examples, the balloon is more compliant along this axial length and less compliant along axial portions beyond this axial length.

Various example kits useful in the sizing of body vessels are described.

An example kit comprises a sizing catheter according to an embodiment and a pressure transducer adapted to be operatively connected to the sizing catheter and to be used for maintaining a pressure, such as a sizing pressure for a particular area of interest in a particular body vessel, on an inner surface of a body vessel at an area of interest in the body vessel.

Various example methods of determining a sizing dimension of a body vessel at an area of interest in the body vessel are described.

An example method of determining a sizing dimension of a body vessel at an area of interest in the body vessel comprises applying a sizing pressure to an inner surface of a body vessel at an area of interest in the body vessel; maintaining the sizing pressure on the inner surface of the body vessel at the area of interest; generating a visual representation of the area of interest; and measuring a dimension of the body vessel at the area of interest on the visual representation to provide the sizing dimension of the body vessel at the area of interest in the body vessel.

Another example method of determining a sizing dimension of a body vessel at an area of interest in the body vessel comprises positioning a pressure-exerting device within a body vessel at an area of interest; using the pressure-exerting device to apply a sizing pressure to an inner surface of the body vessel at the area of interest in the body vessel; maintaining the sizing pressure on the inner surface of the body vessel at the area of interest; generating a visual representation of the area of interest depicting a portion of the pressure-exerting device within the area of interest of the body vessel; and measuring a dimension on the visual representation to provide the sizing dimension of the body vessel at the area of interest in the body vessel.

Another example method of determining a sizing dimension of a body vessel at an area of interest in the body vessel comprises navigating a sizing catheter having a compliant balloon through a body vessel to an area of interest in the body vessel; inflating the compliant balloon to an inflation pressure to apply a corresponding sizing pressure to an inner surface of the body vessel at the area of interest; maintaining the inflation pressure on the compliant balloon; generating a visual representation of the area of interest depicting the inflated compliant balloon within the body vessel at the area of interest; and measuring a dimension of the inflated compliant balloon on the visual representation to provide a sizing dimension of the body vessel at the area of interest in the body vessel.

Various methods of implanting an intraluminal medical device at an area of interest in a body vessel are described.

An example method of implanting an intraluminal medical device at an area of interest in a body vessel comprises applying a sizing pressure to an inner surface of a body vessel at an area of interest in the body vessel; maintaining the sizing pressure on the inner surface of the body vessel at the area of interest; generating a visual representation of the area of interest; measuring a dimension of the body vessel at the area of interest on the visual representation to provide a sizing dimension of the body vessel at the area of interest in the body vessel; and implanting an intraluminal medical device adapted to maintain the sizing dimension within the body vessel. An optional step, performed after the measuring step and before the implanting step if included, comprises confirming that the sizing dimension of the body vessel is within a range of treatment dimensions for the intraluminal medical device.

Another example method of implanting an intraluminal medical device at an area of interest in a body vessel comprises positioning a pressure-exerting device within a body vessel at an area of interest; using the pressure-exerting device to apply a sizing pressure to an inner surface of the body vessel at the area of interest in the body vessel; maintaining the sizing pressure on the inner surface of the body vessel at the area of interest; generating a visual representation of the area of interest depicting a portion of the pressure-exerting device within the area of interest of the body vessel; measuring a dimension on the visual representation to provide the sizing dimension of the body vessel at the area of interest in the body vessel; and implanting an intraluminal medical device adapted to maintain the sizing dimension within the body vessel. An optional step, performed after the measuring step and before the implanting step if included, comprises confirming that the sizing dimension of the body vessel is within a range of treatment dimensions for the intraluminal medical device.

Another example method of implanting an intraluminal medical device at an area of interest in a body vessel comprises navigating a sizing catheter having a compliant balloon through a body vessel to an area of interest in the body vessel; inflating the compliant balloon to an inflation pressure to apply a corresponding sizing pressure to an inner surface of the body vessel at the area of interest; maintaining the inflation pressure on the compliant balloon; generating a visual representation of the area of interest depicting the inflated compliant balloon within the body vessel at the area of interest; measuring a dimension of the inflated compliant balloon on the visual representation to provide a sizing dimension of the body vessel at the area of interest in the body vessel; and implanting an intraluminal medical device adapted to maintain the sizing dimension within the body vessel. An optional step, performed after the measuring step and before the implanting step if included, comprises confirming that the sizing dimension of the body vessel is within a range of treatment dimensions for the intraluminal medical device.

Another example method of implanting an intraluminal medical device at an area of interest in a body vessel comprises applying a first pressure to an inner surface of a body vessel at an area of interest in the body vessel; maintaining the first pressure on the inner surface of the body vessel at the area of interest; generating a visual representation of the area of interest; measuring a dimension of the body vessel at the area of interest on the visual representation to provide a representative dimension of the body vessel at the area of interest in the body vessel; repeating the steps of applying, maintaining generating, and measuring using a second pressure that is different from the first pressure; generating a curve using the first and second pressures and the corresponding first and second measured dimensions; identifying a sizing pressure on the curve; identifying a sizing dimension that corresponds to the sizing pressure on the curve; implanting an intraluminal medical device adapted to maintain the sizing dimension within the body vessel at the area of interest in the body vessel.

Various methods of making an intraluminal medical device are described.

An example method of making an intraluminal medical device comprises applying a sizing pressure to an inner surface of a body vessel at an area of interest in the body vessel; maintaining the sizing pressure on the inner surface of the body vessel at the area of interest; generating a visual representation of the area of interest; measuring a dimension of the body vessel at the area of interest on the visual representation to provide a sizing dimension of the body vessel at the area of interest in the body vessel; and making an intraluminal medical device adapted to exert a pressure of interest, such as the sizing pressure, on an inner surface of the body vessel at the area of interest in the body vessel.

Various methods of making a plurality of intraluminal medical devices are described.

An example method of making a plurality of intraluminal medical devices comprises applying a first pressure to an inner surface of a body vessel at an area of interest in the body vessel; maintaining the first pressure on the inner surface of the body vessel at the area of interest; generating a visual representation of the area of interest; measuring a dimension of the body vessel at the area of interest on the visual representation to provide a representative dimension of the body vessel at the area of interest in the body vessel; repeating the steps of applying, maintaining, generating, and measuring using a second pressure that is different from the first pressure; generating a curve using the first and second pressures and the corresponding first and second measured dimensions; identifying first and second sizing pressures on the curve; identifying a first sizing dimension that corresponds to the first sizing pressure on the curve; identifying a second sizing dimension that corresponds to the second sizing pressure on the curve; making a first intraluminal medical device adapted to exert a first pressure of interest, such as the first sizing pressure, on an inner surface of the body vessel; and making a second intraluminal medical device adapted to exert a second pressure of interest, such as the second sizing pressure, on an inner surface of the body vessel.

Additional understanding of these and other example interventional medical devices, kits, and methods can be obtained by review of the detailed description of selected examples, below, and the referenced drawings.

### DESCRIPTION OF FIGURES

FIG. 1 is a perspective view of an example interventional medical device.

FIG. 2 is a sectional view of a body vessel within which the example interventional medical device illustrated in FIG. 1 is disposed.

FIG. 2A is a side view of a distal portion of an alternative example interventional medical device.

FIG. 2B is a side view of a distal portion of an alternative example interventional medical device.

FIG. 2C is a side view of a distal portion of an alternative example interventional medical device.

FIG. 3 is a schematic illustration of an example kit.

FIG. 4 is a flowchart illustration of an example method of determining a representative dimension of a body vessel at an area of interest in the body vessel.

FIG. 5 is a flowchart illustration of another example method of determining a representative dimension of a body vessel at an area of interest in the body vessel.

FIG. 6 is a flowchart illustration of another example method of determining a representative dimension of a body vessel at an area of interest in the body vessel.

FIG. 7 is a flowchart illustration of an example method of implanting an intraluminal medical device at an area of interest in a body vessel.

FIG. 8 is a flowchart illustration of another example method of implanting an intraluminal medical device at an area of interest in a body vessel.

FIG. 9 is a flowchart illustration of another example method of implanting an intraluminal medical device at an area of interest in a body vessel.

FIG. 10 is a flowchart illustration of another example method of implanting an intraluminal medical device at an area of interest in a body vessel.

FIG. 11 is a flowchart illustration of an example method of making an intraluminal medical device.

FIG. 12 is a flowchart illustration of an example method of making a plurality of intraluminal medical devices.

FIG. 13 illustrates a schematic representation of a pressure-diameter curve and the pressure plateau region of the curve for a generic model body vessel.

FIG. 14 illustrates a simulated pressure-diameter curve for a human vein and shows the expected pressure-plateau region over the range of pressures from about 30 mmHg to about 120 mmHg.

FIG. 15 illustrates a pressure-diameter curve for the right external iliac vein in a porcine model based on experimental data.

FIG. 16 illustrates a simulated pressure-diameter curve, a pressure-diameter curve based on experimental data obtained using methods described herein, and pressure-diameter curves based on study data obtained using IVUS.

### DETAILED DESCRIPTION OF SELECTED EXAMPLES

The following detailed description and the appended drawings describe and illustrate various example interventional medical devices, kits, and methods useful in the sizing of body vessels, methods of implanting intraluminal medical devices, and methods of making intraluminal medical devices. The inclusion of descriptions of these selected examples is not intended to limit the scope of the invention, or its protection, in any manner. The invention is capable of being practiced or carried out in various ways and the examples described and illustrated herein are not considered exhaustive or limiting. Rather, the description and illustration of these examples are provided to enable one skilled in the art to make and use interventional medical devices, such as sizing catheters, and kits, and to perform methods, useful in the sizing of body vessels, and to perform methods of implanting an intraluminal medical device at an area of interest in a body vessel and methods of making intraluminal medical devices.

As used herein, the term “body vessel” refers to a tubular biological structure located within an animal, such as a human being, and having a wall defining a lumen. Examples of body vessels include, but are not limited to, blood vessels, such as arteries and veins, lymphatic vessels, digestive vessels, including the esophagus, the large intestine, and the small intestine, and airway vessels, including the trachea.

As used herein, the term “representative dimension of a sizing dimension” refers to a dimension of an interventional medical device, such as a sizing catheter, or a component of an interventional medical device, such as a balloon, that is measured while the interventional medical device or component of an interventional medical device applies a pressure, such as a sizing pressure, to an inner surface of a body vessel within an area of interest.

As used herein, the term “sizing dimension” refers to a dimension of a body vessel determined at a sizing pressure for the body vessel. A sizing dimension can be an orthogonal inner diameter of a body vessel extending on a plane that is perpendicular to a lengthwise axis of the body vessel.

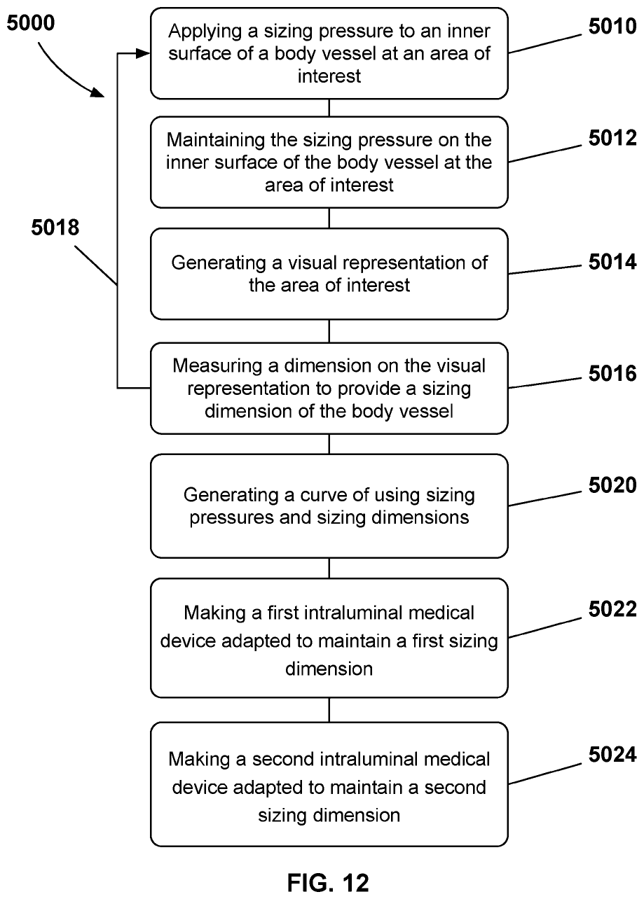

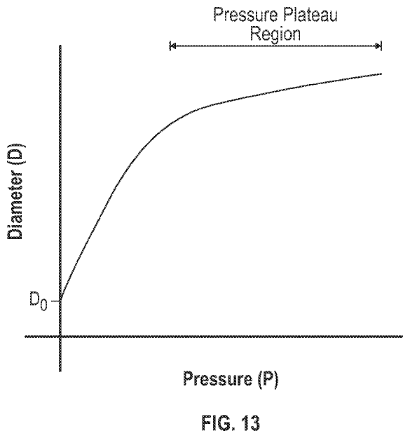

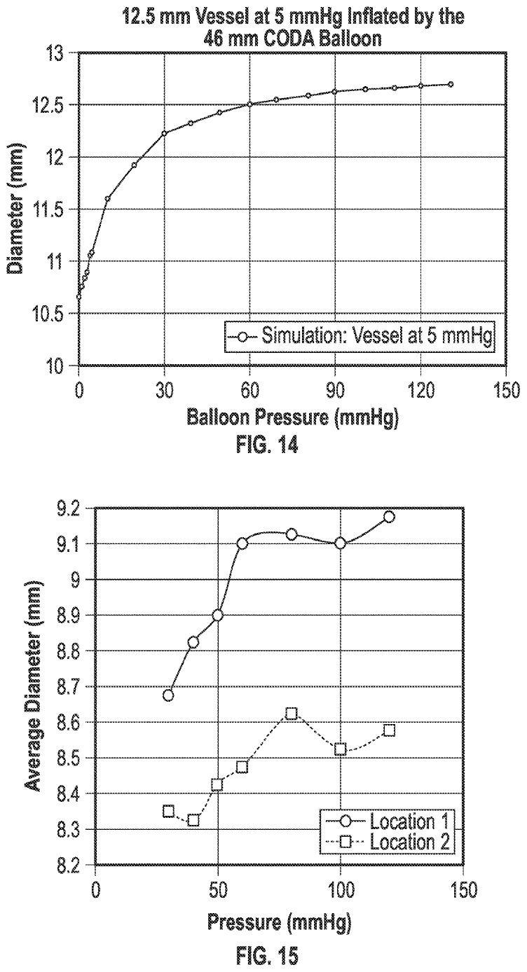

As used herein, the term “sizing pressure” refers to a known constant pressure that is applied to an inner surface of a body vessel in the sizing of the body vessel. A sizing pressure can be a pressure within a range of pressures over which an increase in pressure results in a minimal increase in the diameter of a body vessel and does not result in permanent structural alteration of the body vessel. A sizing pressure also can be a pressure that is below a pressure that results in a permanent change to the structure of the body vessel in response to the pressure being applied to an inner surface of the body vessel. A sizing pressure also can be a pressure located within the pressure plateau region of a pressure-diameter curve showing vessel diameter over a range of known constant pressures for a particular type of body vessel, a specific location within a particular type of body vessel, or specific body vessel, a specific body vessel within a specific animal, or any other identifiable body vessel type, location within a body vessel, or specific body vessel. FIG. 13 illustrates a schematic representation of a pressure-diameter curve and the pressure plateau region of the curve for a generic model body vessel. FIG. 14 illustrates a simulated pressure-diameter curve for a human vein and shows the expected pressure-plateau region over the range of pressures from about 30 mmHg to about 120 mmHg. The rate of change of diameter between successive pressure increments in the plot is less than or equal to about 1 mm over this pressure plateau region. Different vessels are expected to have different pressure plateau values based on the mechanical properties of the vessel, but are expected to demonstrate a vessel-specific pressure plateau region over a range of pressures. FIG. 15, for example, illustrates a pressure-diameter curve of the right external iliac vein in a porcine model based on fluoroscopy. Average vein diameter was measured at sizing catheter pressures in the range of 30-120 mmHg. At each pressure, the vein was imaged in four planes. The vein diameter was measured in each plane and the four values observed at each pressure were averaged to obtain the reported value. Further, measurements were taken at two locations along the length of the sizing catheter. For this body vessel in this model, the expected pressure-plateau region occurs over the range of pressures from about 60 mmHg to about 120 mmHg. A sizing pressure also can be a pressure that results in in a sizing dimension used to choose a particular medical device at or above which the medical device does not migrate under normal physiologic conditions and that is below a pressure that results in a permanent change to the structure of the body vessel. A sizing pressure also can be a pressure that results in in a sizing dimension used to choose a particular medical device at which the medical device is expected to have optimal performance for the particular medical device within a particular type of body vessel, a specific body vessel, a specific body vessel within a specific animal, or any other identifiable body vessel type, location within a body vessel, or specific body vessel.

As used herein, the term “treatment dimension” and “range of treatment dimensions” refers to a dimension or a range of dimensions, as appropriate, at which a medical device is designed and intended to function.

Pressure within a body vessel controls distension of the body vessel. As pressure in a body vessel increases, the relative amount of distension of the body vessel decreases. A pressure exists at which a body vessel achieves full distention; actually achieving full distention, however, risks damaging the body vessel. As defined above, a sizing pressure exists at which further distension of the vessel with increased pressure is inconsequential to device sizing, and the vessel is not damaged.

The inventors have determined that sizing a body vessel at a sizing pressure provides sizing related information, such as a sizing dimension, suitable for use in various methods, such as methods of implanting an intraluminal medical device, while avoiding pressures that may damage the body vessel during the sizing process. To measure a dimension of a body vessel, such as an inner diameter, while not damaging the body vessel, the relationship between the distention of the body vessel and the pressure applied within the body vessel must be known. Applying a sizing pressure to the body vessel wall enables imaging at the appropriate vessel distension to obtain a sizing dimension for sizing a device without damaging the body vessel.

While there are many devices that have been developed to dilate vessels to alter the structure of the body vessel or occlude the body vessel, and many of these devices measure internal device pressure, a need exists for a device that is intended to distend the body vessel with a sizing pressure to determine the sizing dimension of the body vessel for device implantation. Existing devices and methods for sizing vessels are not intended to apply a sizing pressure to obtain the sizing dimension to appropriately size a device.

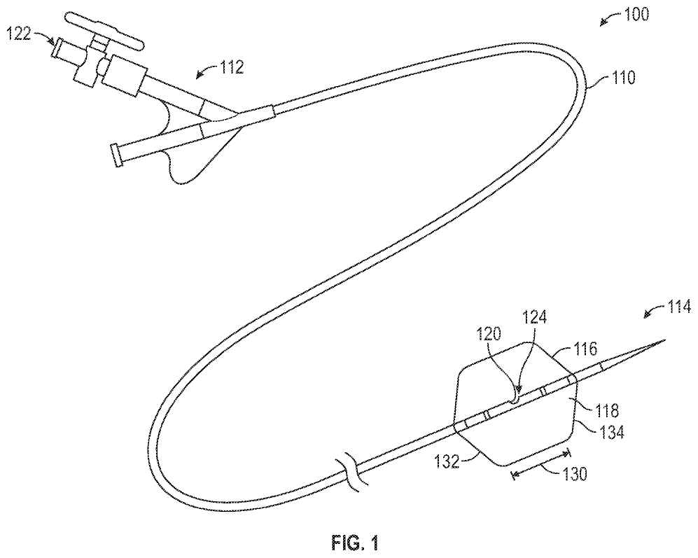

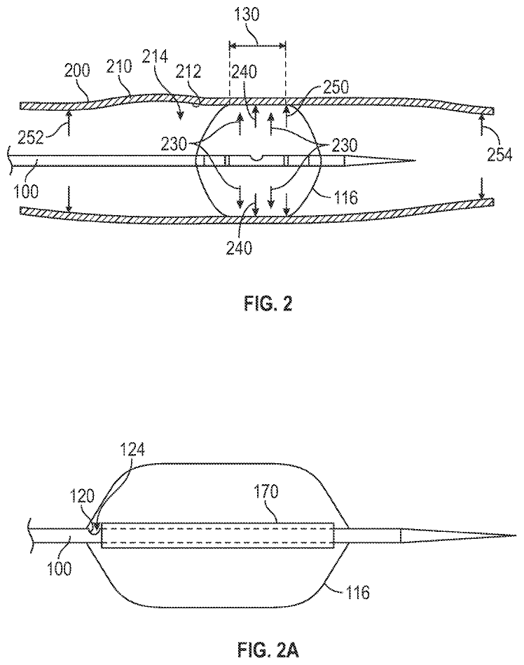

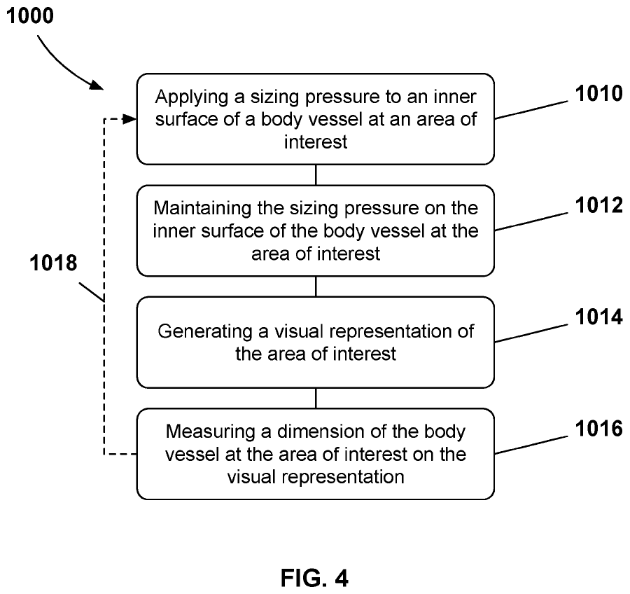

FIG. 1 illustrates an example interventional medical device 100 useful in the sizing of body vessels. FIG. 2 illustrates the interventional medical device 100 disposed in the lumen 214 of a body vessel 200 during performance of a method according to an example, such as method 2000 described below and illustrated in FIG. 5.

Interventional medical device 100 is a sizing catheter. The interventional medical device 100 includes an elongate main body 110 having a proximal end 112 and a distal end 114. A balloon 116 is disposed on the main body 110 adjacent the distal end 114 and defines a cavity 118. The main body 110 defines an inflation lumen 120 having a proximal opening 122 on or near the proximal end 112 of the main body 110 and a distal opening 124 disposed within the cavity 118 of the balloon 116. The balloon 116 is movable between an inflated configuration and a deflated configuration by passing fluid through the inflation lumen 120 and into the cavity 118 and withdrawing fluid from the cavity 118 and through the inflation lumen 120.

The balloon 116 has an axial length 130 along which the pressure transmitted to tissue contacting the balloon 116 is known for at least a single nominal pressure, and advantageously for a range of nominal pressures. As best illustrated in FIG. 1, this axial length 130 can be a portion of the full axial length of the balloon 116. In some embodiments, such as the embodiment illustrated in FIG. 1, the wall of the balloon 116 has a thickness along this axial length 130 that is less than a thickness at the ends 132, 134 disposed axially beyond the axial length 116. This structural configuration is advantageous at least because it provides a balloon 116 that is more compliant along the axial length 130, allowing the balloon to better conform to vessel walls along this axial length 116, and less compliant along the ends of the balloon 116 beyond the axial length 130.

In some embodiments, the balloon 116 is formed such that it has a compliance that is greater than the compliance of a body vessel within which the catheter 100 is intended to be used. For example, the compliance of the balloon can be a multiple of the compliance of a body vessel within which the catheter 100 is intended to be used, such as two times (2×) the compliance of a body vessel within which the catheter 100 is intended to be used, three times (3×) the compliance of a body vessel within which the catheter 100 is intended to be used, five times (5×) the compliance of a body vessel within which the catheter 100 is intended to be used, and ten times (10×) the compliance of a body vessel within which the catheter 100 is intended to be used.

In some embodiments, the balloon 116 has a maximum inflated diameter that is greater than the expected inner diameter of a body vessel within which the catheter 100 is intended to be used. For example, the balloon can have a maximum inflated diameter that is a multiple of the expected inner diameter of a body vessel within which the catheter 100 is intended to be used, such as two times (2×) the expected inner diameter of a body vessel within which the catheter 100 is intended to be used, three times (3×) the expected inner diameter of a body vessel within which the catheter 100 is intended to be used, four times (4×) the expected inner diameter of a body vessel within which the catheter 100 is intended to be used, and five times (5×) the expected inner diameter of a body vessel within which the catheter 100 is intended to be used. In one particular example, a catheter intended to be used in a body vessel expected to have an inner diameter of 12 mm includes a balloon having a maximum inflated diameter of 48 mm.

In some embodiments, the catheter includes a pressure release valve that can be opened to prevent application of a pressure greater than the sizing pressure to the body vessel. In these examples, the pressure release valve can be on the handle end of the catheter or located on the balloon.

FIG. 2 illustrates the interventional medical device 100 illustrated disposed in a body vessel 200, such as during performance of a method according to the invention. The body vessel 200 includes a vessel wall 210 that defines a lumen 214. The interventional medical device 100 is disposed in the lumen 214. The balloon 116 is in an inflated configuration, inflated to an inflation pressure, represented by arrows 230, which results in sizing pressure, represented by arrows 240, transmitted to the inner surface 212 of the vessel wall 210 of the body vessel 200 along axial length 130 of the balloon 116. The interventional medical device 100 is being operated in a manner that maintains the sizing pressure 240 on the inner surface 216 of the vessel wall 210 for a period of time, providing a sizing dimension 250 of the body vessel 200, which can be measured on a visual representation of the illustrated area of the body vessel 200. Dimensions other than the sizing dimension 250, such as dimensions 252, 254 representing diameters of the body vessel 200 at which the sizing pressure 240 is not applied to the inner surface 212 of the vessel wall 210.

Interventional medical devices according to embodiments can include additional and alternative structural components and arrangements, including components that facilitate use of the interventional medical devices in methods according to embodiments. For example, as illustrated in FIG. 2A, interventional medical device 100 can include a cannula 170, such as a metal cannula, disposed over a portion of the elongate main body 110 within the cavity 118 of the balloon 116. If included, the cannula 170 can be free floating over the portion of the elongate main body 110 within the cavity 118 of the balloon 116, or can be attached to the elongate main body 110 such that the cannula 170 is secured in a fixed position within the cavity 118 within the balloon 116. If attached, any suitable attachment can be used, such as swaging, adhesives, and the like. Distal opening 124 of inflation lumen 120 can be disposed relatively more proximally or distally within the cavity 118 of the balloon 116 with respect to the position illustrated in FIG. 1 to avoid disruption of fluid flow through the distal opening 124 due to the presence of the cannula 170. Inclusion of cannula 170 is considered advantageous at least because the cannula 170 can be made precisely to a specific and known length that can be used as a calibration reference during use of the interventional medical device 100. While markers disposed at a known distance from each other on the elongate main body 110 can be used as a calibration reference, the elongate main body 110 can bend or otherwise distort during inflation of the balloon 116, rendering unknown the distance between the markers and, in turn, eliminating the reliability of the markers as a calibration reference. The presence of cannula 170 prevents, or substantially prevents, such bending, providing a reliable calibration reference through the entire range of potential pressures used in inflating the balloon 116. For at least this reason, inclusion of cannula 116 in interventional medical devices used in the methods described herein is considered particularly advantageous.

Interventional medical devices according to embodiments can include additional and alternative structural components and arrangements that facilitate use of the interventional medical devices in methods according to embodiments with particular imaging modalities. For example, each of FIGS. 2B and 2C illustrates an interventional medical device that is configured for use with intravascular ultrasound (IVUS) imaging. In FIG. 2B, interventional medical device 180 includes an elongate main body 181 the defines a lumen 182 that can accommodate a wireguide and an IVUS catheter 183 having an ultrasound transducer 184. The transducer(s) may be a phased array, rotating single element, linear array, or any other configuration known to one skilled in the art of IVUS catheter design. The IVUS catheter 183 is movable within the lumen 182 such that the transducer 184 can be positioned within the portion 185 of the elongate main body 181 that is disposed within the balloon 186. The material of the elongate main body 110 in this portion 185 is made of an echolucent material to allow for ultrasound signals transmitted and received by the transducer 184 to pass through the portion 185. The balloon 186 can also be made of an echolucent material to facilitate transmission of such signals through the balloon to enable visualization outside of the balloon 186. Examples of suitable echolucent materials include, but are not limited to, methyl methacrylate butadiene styrene copolymer (MBS), acrylonitrile butadiene styrene (ABS), methacrylate-acrylonitrile-butadiene-styrene (MABS), and Styrene butadiene block copolymer (SBC), Polymethylpentene (PMP), Polyethylene (PE), polyamide (e.g., nylon), and polyimide. Alternatively, the balloon 186 can include echogenic features to enable clear visualization of locations on the balloon. A coupling medium 187 is disposed within balloon 186 when inflated to allows for the transmission of ultrasound signals 188 between the different interfaces with minimal signal loss. Coupling medium 187 must have a similar acoustic impedance to balloon 186 and the vessel wall adjacent balloon 186 to allow for maximum transmission of ultrasound signals 188. Suitable coupling mediums include, but are not limited to, biocompatible fluids such as saline, oils such as mineral or soybean oils, alcohols, or other fluids.

In FIG. 2C, interventional medical device 190 includes an elongate main body 191 having a ultrasound transducer 192 integrated into the elongate main body 191 such that the transducer 192 is located within the balloon 193. When the balloon 193 is inflated with the coupling medium 194, ultrasound signals 195 only need be transmitted and/or received through the coupling medium 194 and material of the balloon 193, thus minimizing any potential attenuation of signals. In additional embodiments multiple transducers may be located along the length of the balloon to allow for evaluation of multiple locations simultaneously.

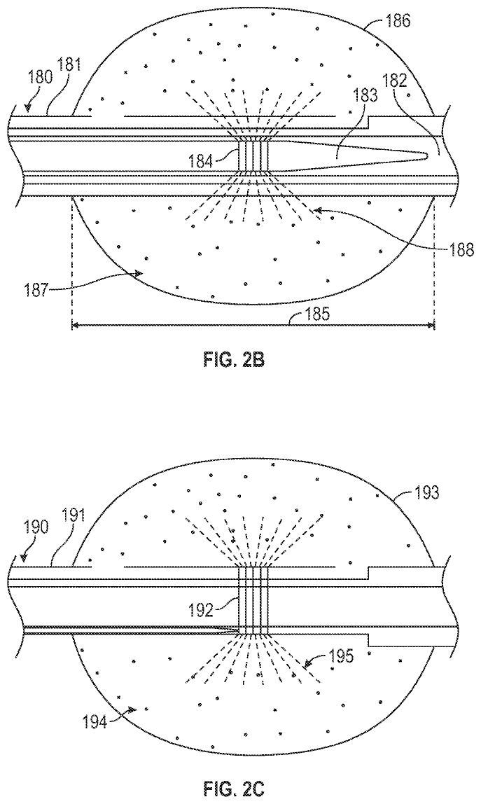

FIG. 3 illustrates a kit 300 useful in the sizing of body vessels. Kit 300 includes sizing catheter 100 described above and illustrated in FIGS. 1 and 2, and a pressure maintaining device 310 adapted to be operatively connected to the sizing catheter and to maintain a pressure of interest, such as a pressure suitable for applying a sizing pressure to an inner surface of a body vessel, in the balloon of the sizing catheter. Instructions for use 312 can also be included in kit 300. In the illustrated embodiment, pressure maintaining device 310 comprises a pressure transducer. Another device suitable for maintaining a pressure of interest in the balloon of the sizing catheter can be included as the pressure maintaining device 310 in a kit according to an embodiment, such as a pressure gauge that correlates pressure and diameter, pressure and volume, or both, a pressure indicator, or other suitable device adapted to maintain a pressure of interest in the balloon of the sizing catheter. Also alternatively, a source of known constant pressure that is adapted to be operatively connected to the sizing catheter for maintaining the pressure of interest in the balloon of the sizing catheter can be included in a kit according to an embodiment. For example, an inflation source of known constant pressure, such as a regulated pressure supply or a defined column of a liquid, such as water. In one embodiment, a kit includes a syringe adapted to be operatively connected to the sizing catheter and that includes indicators for the volume of fluid to inject to achieve a given pressure specified by the manufacturer.

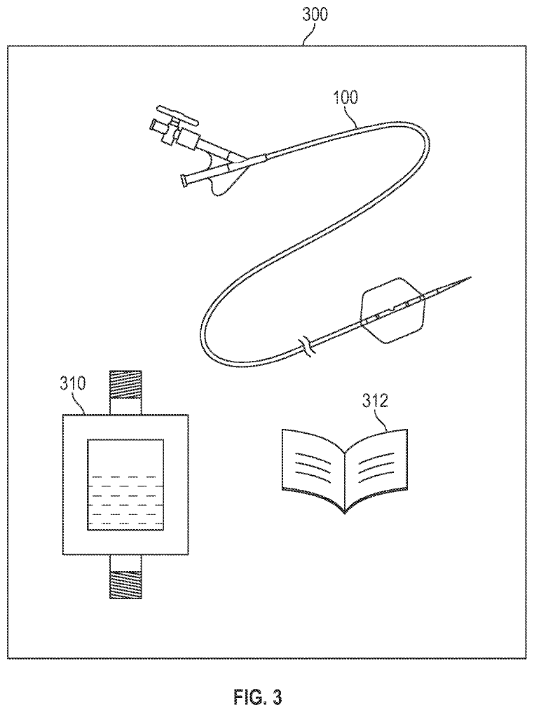

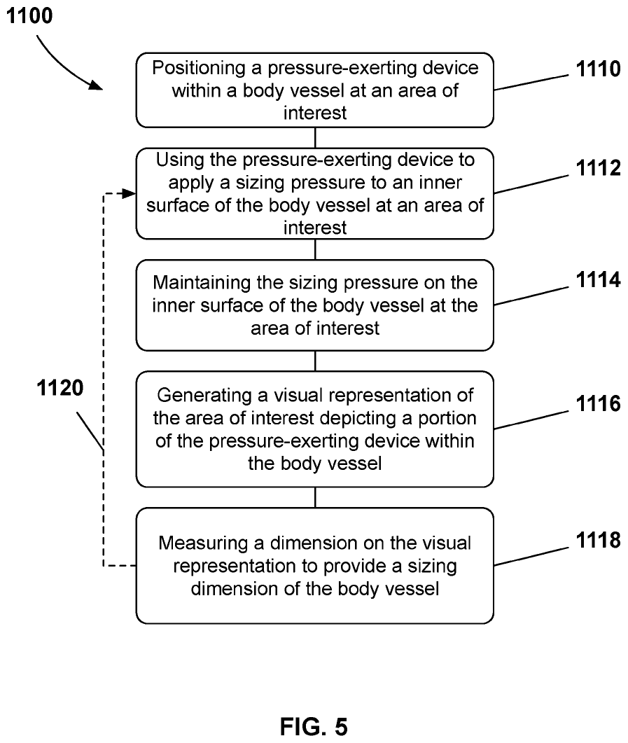

Methods of determining a sizing dimension of a body vessel at an area of interest are provided. Each of FIGS. 4, 5, and 6 illustrates an example method in accordance with this aspect of the invention. Performance of these and other methods in accordance with this aspect of the invention provides a sizing dimension of a body vessel by measuring a dimension on a visual representation of the body vessel while a sizing pressure is maintained on an inner surface of the body vessel. In some example methods, such as method 1000 illustrated in FIG. 4 and described below, the sizing dimension is a dimension of the body vessel, such as an inner diameter of the body vessel that is orthogonal to the longitudinal axis of the body vessel. In other example methods, such as method 1200 illustrated in FIG. 6 and described below, the sizing dimension is a dimension of a pressure-exerting device, such as a sizing catheter, disposed within the body vessel as part of the performance of the method.

An example method of determining a sizing dimension of a body vessel at an area of interest comprises measuring a dimension on a visual representation of the body vessel while a sizing pressure is maintained on an inner surface of the body vessel. In some examples, the sizing dimension is a dimension of the body vessel, such as an orthogonal inner diameter of the body vessel. In other examples, the sizing dimension is a dimension of a pressure-exerting device used in the method for applying the sizing pressure to the inner surface of the body vessel.

FIG. 4 illustrates an example method 1000 of determining a sizing dimension of a body vessel at an area of interest in the body vessel. An initial step 1010 comprises applying a sizing pressure to an inner surface of a body vessel at an area of interest in the body vessel. Another step 1012 comprises maintaining the sizing pressure on the inner surface of the body vessel at the area of interest. Another step 1014 comprises generating a visual representation of the area of interest. Another step 1016 comprises measuring a dimension of the body vessel at the area of interest on the visual representation to provide the sizing dimension of the body vessel at the area of interest in the body vessel.

The method 1000, and indeed all methods according to the invention, can be performed on any suitable body vessel in the body of an animal, including human and non-human animals. Examples of suitable body vessels include, but are not limited to, arteries, veins, lymphatic vessels, digestive tract vessels such as the esophagus, small intestine, and large intestine, airway vessels, and other body vessels. Methods according to the invention are particularly advantageous for performance on compliant body vessels, such as veins and lymphatic vessels, at least because these vessels are challenging to evaluate dimensionally using conventional techniques.

Similarly, the method 1000, and indeed all methods according to the invention, can be performed on any suitable area of interest in a body vessel. Examples of suitable areas of interest include a location of a natural valve within a body vessel, such as the location of a natural venous valve in a vein, a location of an opening to a branch vessel, a location within a body vessel that is adjacent an external anatomical feature or structure within the body, such as an adjacent body vessel. For example, methods according to the invention can be performed at an area in a left common iliac vein of a human that is compressed by the overlying right common iliac artery, such as the compression that occurs in May-Thurner syndrome.

The step 1010 of applying a sizing pressure to an inner surface of a body vessel at an area of interest in the body vessel can be performed using a variety of pressure-exerting devices and techniques. Examples of suitable pressure-exerting devices for applying a sizing pressure include, but are not limited to, sizing catheters that include a balloon, including sizing catheters that include a compliant balloon, such as the sizing catheter illustrated in FIGS. 1 and 2 and described above, expandable intraluminal baskets, and other pressure-exerting intraluminal medical devices. Sizing catheters that include a compliant balloon are considered particularly advantageous at least because their ability to stretch allows the balloon to fully conform to and evenly distribute pressure along the inner surface of the body vessel adjacent the balloon or portion of the balloon. Sizing catheters used in the method 1000 that include a balloon advantageously include a component for controlling inflation and deflation, and, therefore, pressure, of the balloon, such as a valve that controls passage of inflation fluid into and out of the inflation lumen and balloon, a pressure transducer that includes a valve and a controller for setting a specific desired pressure, or other suitable component. Examples of suitable techniques for applying a sizing pressure to an inner surface of a body vessel include, but are not limited to, inflating the balloon of a sizing catheter by passing an inflation fluid, such as a physiological saline or other liquid or carbon dioxide or other suitable gas, through an inflation lumen of the catheter into the balloon to cause inflation of the balloon. Advantageously, sizing catheters used in the method 1000 that include a balloon are characterized so that the relationship between the inflation pressure and the sizing pressure transmitted to the tissue is known along a specified length of the balloon. A rate of change in this relationship over time, if any, is also advantageously characterized for any sizing catheter used in the method 1000.

In performing the step 1010 of applying a sizing pressure, the sizing pressure can be any suitable sizing pressure and a skilled artisan will be able to select an appropriate or desirable sizing pressure for a method according to a particular example based on various considerations, including the type of body vessel on which the method is being performed, the nature of the location of interest within the body vessel at which the method is being performed, and any anticipated use of the sizing dimension to be determined by performance of the method. Examples of suitable pressures that can be used as sizing pressures in methods according to examples include, but are not limited to, a maximum pressure the inner surface of the body vessel at the area of interest is expected to be exposed to naturally, a pressure exerted by an intraluminal medical device adapted for implantation at the point of interest in the body vessel, such as by the radial force exerted by a self-expandable frame in a stent, a frame in a stent that requires input of force to achieve radial expansion, such as a balloon expandable stent, a support frame in a valve device, or other intraluminal medical device, and other pressures. Advantageously, the sizing pressure is lower than a pressure that results in dilation of the body vessel accompanied by a permanent structural alteration of the body vessel. For veins, specific examples of suitable pressures that can be used as a sizing pressure in methods according to examples include, but are not limited to, a pressure that is greater than 0 mmHg and is less than about 1000 mmHg, a pressure that is greater than 0 mmHg and is less than about 750 mmHg. a pressure that is greater than 0 mmHg and is less than about 500 mmHg, a pressure that is greater than 0 mmHg and is less than about 250 mmHg, a pressure that is greater than 0 mmHg and is less than about 200 mmHg, a pressure that is greater than 0 mmHg and is less than about 150 mmHg, a pressure that is greater than 0 mmHg and is less than about 130 mmHg, a pressure that is greater than 0 mmHg and is less than about 120 mmHg, a pressure equal to or less than about 10 mmHg, a pressure equal to or less than about 20 mmHg, a pressure equal to or less than about 30 mmHg, a pressure equal to or less than about 40 mmHg, a pressure equal to or less than about 50 mmHg, a pressure equal to or less than about 60 mmHg, a pressure equal to or less than about 70 mmHg, a pressure equal to or less than about 80 mmHg, a pressure equal to or less than about 90 mmHg, a pressure equal to or less than about 100 mmHg, a pressure equal to or less than about 110 mmHg, a pressure equal to or less than about 120 mmHg, a pressure equal to or less than about 130 mmHg, a pressure equal to or less than about 140 mmHg, and a pressure equal to or less than about 150 mmHg.

The step 1012 of maintaining the sizing pressure on the inner surface of the body vessel at the area of interest can be performed using a technique suitable for any device used in the step 1010 of applying a sizing pressure. For example, if the device includes a valve that controls the passage of inflation fluid into a balloon, such as in a sizing catheter, the valve can be closed once the sizing pressure is achieved in the balloon such that no further inflation fluid passes into the balloon and no inflation fluid within the balloon or the inflation lumen of the sizing catheter is removed from the balloon or the inflation lumen. For balloons that exhibit a degree of creep, fluid can be added as needed to accommodate the creep and maintain the sizing pressure. It is noted that the step 1012 of maintaining the sizing pressure, in this and all methods, can be performed by maintaining, over a period of time, the relevant sizing pressure or a range of pressures that includes the relevant sizing pressure. Also, maintaining the sizing pressure can comprise maintaining a set rate of change for the pressure in a system such that a time at which the sizing pressure was present can be determined. Also, balloons constructed from viscoelastic materials, or other materials that have a time-dependent mechanical response under relevant conditions, may exhibit a bit of creep, which may result in pressure decreases over time. Accordingly, the step of maintaining the sizing pressure can be performed by adding fluid to accommodate this creep.

The step 1014 of generating a visual representation of the area of interest can be performed using any medical imaging modality adapted for generating a visual representation of the area of interest of the body vessel. A skilled artisan will be able to select a suitable medical imaging modality for use in a method according to a particular example based on various considerations, including the body vessel and the area of interest within the body vessel, patient-specific considerations such as X-ray and MRI exposure concerns, and other considerations. Examples of suitable medical imaging modalities include, but are not limited to, radiography, X-ray computed tomography, fluoroscopy, venography, ultrasound, and magnetic resonance imaging (MRI). For veins, venography that includes injection of contrast into the balloon is considered particularly advantageous at least because the balloon becomes fully apposed to the inner surface of the body vessel and, therefore, shows the inner surface of the body vessel in resultant images.

For the step of 1014, the visual representation can comprise any suitable visual image, including an image recorded on film, such as an X-ray image, a digital still image, such as a digital still image stored on a computer hard drive, random access memory, flash memory, such as a solid state drive or USB flash drive, or other suitable digital storage medium, a series of multiple such digital still images, such as a series of still images captured over a period of time, a series of multiple such digital still images captured from multiple angles, such as a range of angles, and a digital video stored on a suitable digital storage medium, such as those recited above. The type and number of visual images generated during the performance of step 1014 may depend on the medical imaging modality used. For example, for medical imaging modalities that are not three dimensional, it can be advantageous to capture multiple images of the area of interest from multiple angles. These multiple images can then be used in the step 1016 of measuring a dimension of the body vessel, for example, as described below.

Advantageously, the step 1014 of generating a visual representation of the area of interest is performed while the step 1012 of maintaining the sizing pressure on the surface of the body vessel at the area of interest is performed. This temporal relationship of the performance of these steps 1012, 1014 assures that the step 1016 of measuring a dimension on the visual representation provides the sizing dimension of the body vessel at the area of interest while the sizing pressure is maintained on the inner surface of the body vessel at the area of interest.

The step 1016 of measuring a dimension of the body vessel at the area of interest on the visual representation can be performed using any suitable technique and any suitable tools, including manual tools and software. For example, if the medical imaging modality used in step 1014 includes digital imaging capabilities and image analysis software, a measuring tool in the image analysis software can be used to set opposing points on the inner surface of the body vessel, generate a line between the opposing points, and calculate a distance between the points in scale relevant to the body vessel, and not necessarily the image. Any suitable dimension of the body vessel can be measured in step 1016. Advantageously, the dimension is a width of the body vessel extending from a first point on the inner surface of the body vessel to a second point on the inner surface of the body vessel that is opposite the first point with respect to the axis of the body vessel at the area of interest. In some examples, the sizing pressure is a pressure that circularizes the body vessel at the area of interest and the sizing dimension is an inner diameter of the body vessel extending between first and second points on the inner surface of the body vessel and on a plane that is perpendicular to the axis of the body vessel at the area of interest. In other examples, the sizing pressure is a pressure that less than a pressure that fully circularizes the body vessel at the area of interest. For example, in methods in which a sizing catheter is used in the step 1010 of applying a sizing pressure to an inner surface of a body vessel at an area of interest in the body vessel, an oversized balloon (i.e., a balloon with redundant material), relative to an expected natural maximum size of the body vessel, can be less than fully inflated using a pressure that allows the balloon to conform to the inner surface of the body vessel without expanding the body vessel or even altering the surface area of the inner surface of the body vessel in any substantial manner. These examples can be beneficial when the area of interest includes one or more tributary vessels of the body vessel as the less than fully inflated balloon will extend slightly into the lumen of the tributary vessel or vessels as it conforms to the inner surface of the body vessel, allowing the presence, dimensions, or both of the one or more tributary vessels to be incorporated into the step 1016 of measuring a dimension of the body vessel at the area of interest on the visual representation.

Multiple dimensions can be measured during performance of step 1016 if appropriate. For example, as discussed above, it can be advantageous in the performance of step 1014 to capture multiple images of the area of interest from multiple angles when medical imaging modalities that are not three dimensional are used. With fluoroscopy, for example, multiple two-dimensional images can be generated during performance of step 1014. Then, performance of this step 1016 can comprise measuring a dimension of the body vessel on each of the images and processing the multiple dimensions obtained, such as by calculating an average of the multiple dimensions, to provide the sizing dimension of the body vessel at the area of interest in the body vessel.

In method 1000, steps 1010, 1012, 1014, and 1016 can be repeated any desired number of times, as represented by optional step 1018. Repeating steps 1010, 1012, 1014, and 1016 can be advantageous if it is desirable to determine multiple dimensions of the body vessel or to determine the sizing pressure. If step 1018 is included, the sizing pressure in each cycle of steps 1010, 1012, 1014, and 1016 can be the same or different than the sizing pressure in a prior cycle of steps 1010, 1012, 1014, and 1016. Using a different sizing pressure in a subsequent cycle of steps 1010, 1012, 1014, and 1016 is advantageous at least because completion of a method in which different pressures of interest are used provides multiple dimensions of the body vessel at different pressures of interest, enabling determination of the sizing pressure and thus the sizing dimension or evaluation of the body vessel at different areas of interest.

Completion of the method provides the dimension, which is a sizing dimension of the body vessel at the area of interest in the body vessel.

FIG. 5 illustrates another example method 1100 of determining a sizing dimension of a body vessel at an area of interest in the body vessel. The method 1100 is similar to the method 1000 described above and illustrated in FIG. 4, except as detailed below.

An initial step 1110 comprises positioning a pressure-exerting device within a body vessel at an area of interest. Another step 1112 comprises using the pressure-exerting device to apply a sizing pressure to an inner surface of the body vessel at the area of interest in the body vessel. Another step 1114 comprises maintaining the sizing pressure on the inner surface of the body vessel at the area of interest. Another step 1116 comprises generating a visual representation of the area of interest depicting a portion of the pressure-exerting device within the area of interest of the body vessel. Another step 1118 comprises measuring a dimension on the visual representation to provide the sizing dimension of the body vessel at the area of interest in the body vessel.

The step 1110 of positioning a pressure-exerting device within a body vessel at an area of interest can be performed using a variety of devices and techniques, including sizing catheters, such as a catheter that includes a compliant balloon. The positioning can be performed using technique suitable for the pressure-exerting device used in the step 1110. For example, if a sizing catheter is used, the sizing catheter can be inserted into an opening into the body vessel and passed into the body vessel to navigate the balloon to the area of interest. Navigation in this example can be performed over a previously-placed guidewire or without the aid of a guidewire.

The step 1112 of using the pressure-exerting device to apply a sizing pressure can be performed using a technique suitable for the pressure-exerting device used in step 1110. For example, if a sizing catheter is used in step 1110, step 1112 can be performed by inflating the balloon to a pressure of interest that applies the sizing pressure to the inner surface of the body vessel at the area of interest. The pressure to which the balloon is inflated can be the sizing pressure or different than the sizing pressure as not all of the inflation pressure of the balloon may be transmitted to the inner surface of the body vessel.

The step 1114 of maintaining the sizing pressure on the inner surface of the boy vessel can be performed in the same manner as step 1012 in method 1000, as described above.

The step 1116 of generating a visual representation of the area of interest can be performed in the same manner as step 1014 in method 1000, as described above, except as described below. In this method 1100, step 1116 is performed such that at least a portion of the pressure-exerting device used in step 1010 is depicted in the visual representation. For example, if the pressure-exerting device comprises a sizing catheter, step 1116 is performed such that at least the balloon of the sizing catheter is depicted in the visual representation. Advantageously, the step 1116 of generating a visual representation of the area of interest is performed while the step 1014 of maintaining the sizing pressure on the surface of the body vessel at the area of interest is performed. This temporal relationship of the performance of these steps 1114, 1116 assures that the step 1118 of measuring a dimension on the visual representation provides a sizing dimension of the body vessel at the area of interest while the sizing pressure is maintained on the inner surface of the body vessel at the area of interest.

The step 1118 of measuring a dimension on the visual representation can be performed in the same manner as step 1016 n method 1000, as described above, except as described below. In this method 1100, the dimension can either be a dimension of the body vessel, such as an inner diameter of the body vessel on a plane that is perpendicular to the axis of the body vessel at the area of interest, an inner diameter of the body vessel on a plane that is not perpendicular to the axis of the body vessel at the area of interest, or a dimension of the pressure-exerting device, such as a width of the inflated balloon measured on a plane that is perpendicular to the axis of the body vessel at the area of interest, on a plane that is perpendicular to the axis of the sizing catheter along the length of the balloon, or on a plane that is perpendicular to both the axis of the body vessel at the area of interest and that is perpendicular to the axis of the sizing catheter along the length of the balloon.

In method 1100, steps 1112, 1114, 1116, and 1118 can be repeated any desired number of times, as represented by optional step 1120. Repeating steps 1112, 1114, 1116, and 1118 can be advantageous if it is desirable to determine multiple dimensions of the body vessel. If step 1120 is included, the sizing pressure in each cycle of steps 1112, 1114, 1116, and 1118 can be the same or different than the sizing pressure in a prior cycle of steps 1112, 1114, 1116, and 1118. Using a different sizing pressure in a subsequent cycle of steps 1112, 1114, 1116, and 1118 is advantageous at least because completion of a method in which different pressures of interest are used provides multiple dimensions of the body vessel at different pressures of interest, enabling determination of the sizing pressure and thus the sizing dimension or evaluation of the body vessel at different areas of interest.

Completion of the method 1100 provides one or more dimensions, each of which is a dimension of the body vessel at the area of interest in the body vessel.

FIG. 6 illustrates another example method 1200 of determining a sizing dimension of a body vessel at an area of interest in the body vessel. The method 1200 is a specific application of method 1000 described above and illustrated in FIG. 4.

An initial step 1210 comprises navigating a sizing catheter having a compliant balloon through a body vessel to an area of interest in the body vessel. Another step 1212 comprises inflating the compliant balloon to an pressure of interest to apply a corresponding sizing pressure to an inner surface of the body vessel at the area of interest. Another step 1214 comprises maintaining the pressure of interest on the compliant balloon. Another step 1216 comprises generating a visual representation of the area of interest depicting the inflated compliant balloon within the body vessel at the area of interest. Another step 1218 comprises measuring a dimension of the inflated compliant balloon on the visual representation to provide a sizing dimension of the body vessel at the area of interest in the body vessel.

Advantageously, the step 1216 of generating a visual representation of the area of interest is performed while the step 1214 of maintaining the inflation pressure within the balloon at the area of interest is performed. This temporal relationship of the performance of these steps 1214, 1216 assures that the step 1218 of measuring a dimension of the inflated compliant balloon on the visual representation provides a dimension of the body vessel at the area of interest while the inflation pressure is maintained within the balloon at the area of interest.

Method 1200 is particularly advantageous for performance on relatively compliant body vessels, such as veins and lymphatic vessels. The method utilizes a sizing catheter having a compliant balloon. Furthermore, the compliant balloon exerts a sizing pressure on an inner surface of a body vessel for a particular inflation pressure within the balloon. The sizing pressure for any given inflation pressure can be the same as or less than the inflation pressure and can be determined empirically for a particular inflation pressure or over a range of inflation pressures. Alternatively, a balloon for which exertion pressure(s) have been determined by another, such as a manufacturer of the sizing catheter, can be used. Also alternatively, a balloon for which the relationship between balloon volume(s) and sizing pressure(s) is determined empirically or by another, such as a manufacturer of the sizing catheter, can be used.

In method 1200, steps 1212, 1214, 1216, and 1218 can be repeated any desired number of times, as represented by optional step 1220. Repeating steps 1212, 1214, 1216, and 1218 can be advantageous if it is desirable to determine multiple dimensions of the body vessel to determine a sizing dimension. If step 1220 is included, the sizing pressure in each cycle of steps 1212, 1214, 1216, and 1218 can be the same or different than the sizing pressure in a prior cycle of steps 1212, 1214, 1216, and 1218. Using a different sizing pressure in a subsequent cycle of steps 1212, 1214, 1216, and 1218 is advantageous at least because completion of a method in which different pressures of interest are used to provide a sizing pressure thus providing a sizing dimension of the body vessel.

A determination of one or more dimensions of a body vessel based on pressure applied to an internal surface of the body vessel can be part of other methods, such as methods of implanting an intraluminal medical device, methods of making intraluminal medical devices, and others.

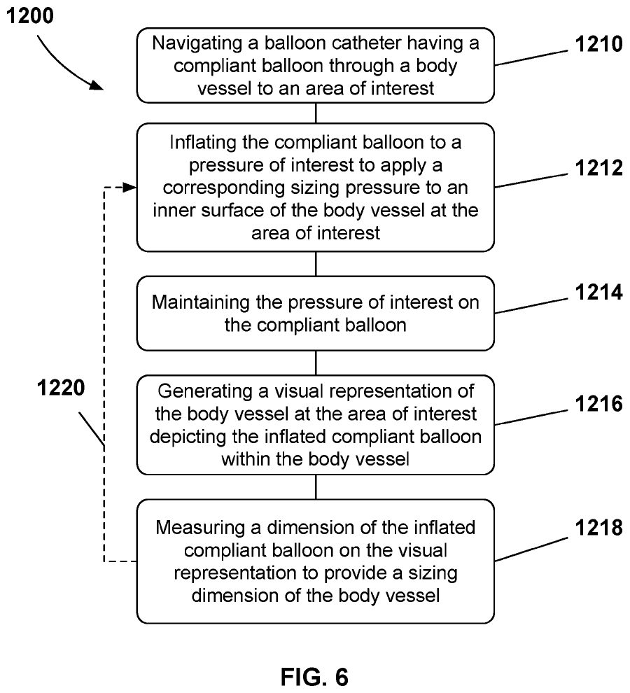

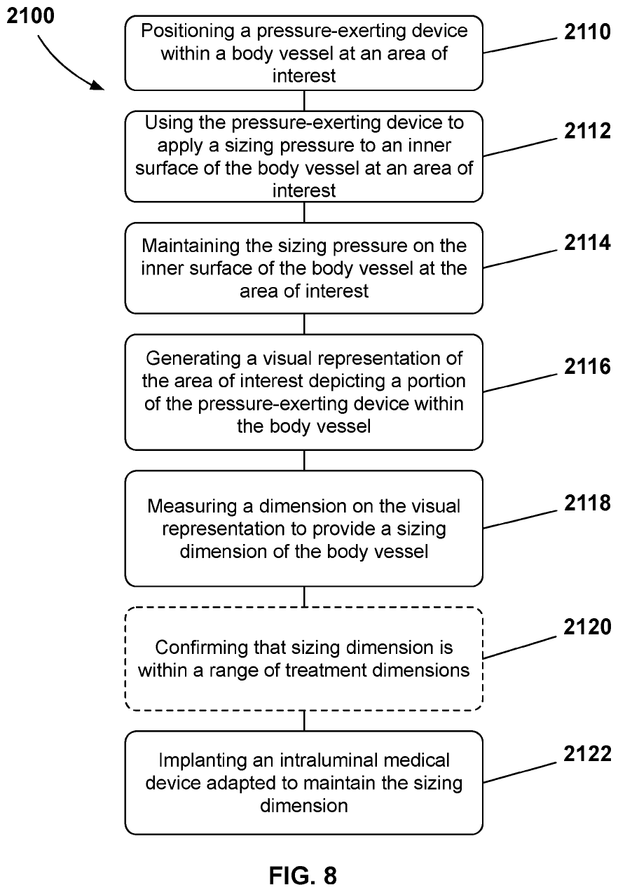

Accordingly, methods of implanting an intraluminal medical device at an area of interest in a body vessel are provided. Each of FIGS. 7, 8, 9, and 10 illustrates an example method in accordance with this aspect of the invention. Performance of these and other methods in accordance with this aspect of the invention results in the implantation of an intraluminal medical device appropriately sized to prevent migration after measuring the sizing dimension on a visual representation of the body vessel while the sizing pressure is maintained on an inner surface of the body vessel and confirming that the sizing dimension is within a range of desired treatment dimensions of the intraluminal medical device. In some example methods, such as method 2000 illustrated in FIG. 7 and described below, the sizing dimension is a dimension of the body vessel, such as an orthogonal inner diameter of the body vessel. In other example methods, such as method 2200 illustrated in FIG. 9 and described below, the dimension is a dimension of a pressure-exerting device, such as a compliant balloon of a sizing catheter disposed within the body vessel as part of the performance of the method.

An example method of implanting an intraluminal medical device at an area of interest in a body vessel comprises implanting an intraluminal medical device onto the inner surface of a body vessel after measuring a sizing dimension on a visual representation of the body vessel while the sizing pressure is maintained on an inner surface of the body vessel at the area of interest and confirming that the sizing dimension is within a range of desired treatment dimensions. In some examples, the sizing dimension is a dimension of the body vessel, such as an orthogonal inner diameter of the body vessel measured while the sizing pressure is maintained on the inner surface of the body vessel at the area of interest. In other examples, the sizing dimension is a dimension of a pressure-exerting device used in the method for applying the sizing pressure to, and maintaining the sizing pressure on, the inner surface of the body vessel.

FIG. 7 illustrates an example method 2000 of implanting an intraluminal medical device at an area of interest in a body vessel. The method 2000 is similar to the method 1000 illustrated in FIG. 4, with the addition of steps 2018, 2020 required for implanting an intraluminal medical device. Accordingly, an initial step 2010 comprises applying a sizing pressure to an inner surface of a body vessel at an area of interest in the body vessel. Another step 2012 comprises maintaining the sizing pressure on the inner surface of the body vessel at the area of interest. Another step 2014 comprises generating a visual representation of the area of interest. Another step 2016 comprises measuring a dimension of the body vessel at the area of interest on the visual representation to provide a sizing dimension of the body vessel at the area of interest in the body vessel. Another step 2018, which is optional, comprises confirming that the sizing dimension of the body vessel provided by step 2016 is within a range of treatment dimensions for the intraluminal medical device. Another step 2020 comprises implanting an intraluminal medical device adapted to maintain the sizing dimension of the inner surface of a body vessel.

Advantageously, step 2014 of generating a visual representation of the area of interest is performed while the step 2012 of maintaining the sizing pressure on the surface of the body vessel at the area of interest is performed. This temporal relationship of the performance of these steps 2012, 2014 assures that the step 2016 of measuring a dimension on the visual representation provides the sizing dimensions of the body vessel at the area of interest while the sizing pressure is maintained on the inner surface of the body vessel at the area of interest.

For step 2018, if included, the range of treatment dimensions of the intraluminal medical device can comprise a range based on prior performances of the method, known or expected values, or another suitable source.

Any suitable intraluminal medical device can be used in the performance of step 2020. The device need only be biocompatible and capable of being implanted in a body vessel of the same nature of the body vessel on which the method is being performed. The specific type, size, and configuration of the intraluminal medical device used in step 2020 in a specific method will depend on various considerations, including the nature of the body vessel and desired outcomes of the method. Examples of types of intraluminal medical devices that can be used in the performance of step 2020 include, but are not limited to, stents, stent grafts, valve devices, such as prosthetic venous valves and prosthetic heart valves, filters, occluders, distal protection devices, and other implantable intraluminal medical devices. Intraluminal medical devices that include a self-expandable support frame, such as self-expandable stents, valve devices that include a self-expandable support frame, and other intraluminal medical devices that include a self-expandable frame are considered particularly advantageous for inclusion in performance of step 2020.

Step 2020 can be performed using conventional techniques for implanting the particular intraluminal medical device used in the step 2020, including any ancillary devices necessary or desirable for implanting the intraluminal medical device. For example, the intraluminal medical device can be navigated to the area of interest in the body vessel on a delivery catheter using conventional insertion and navigation techniques and subsequently deployed from the delivery catheter using conventional techniques suitable for the intraluminal medical device and the delivery catheter.

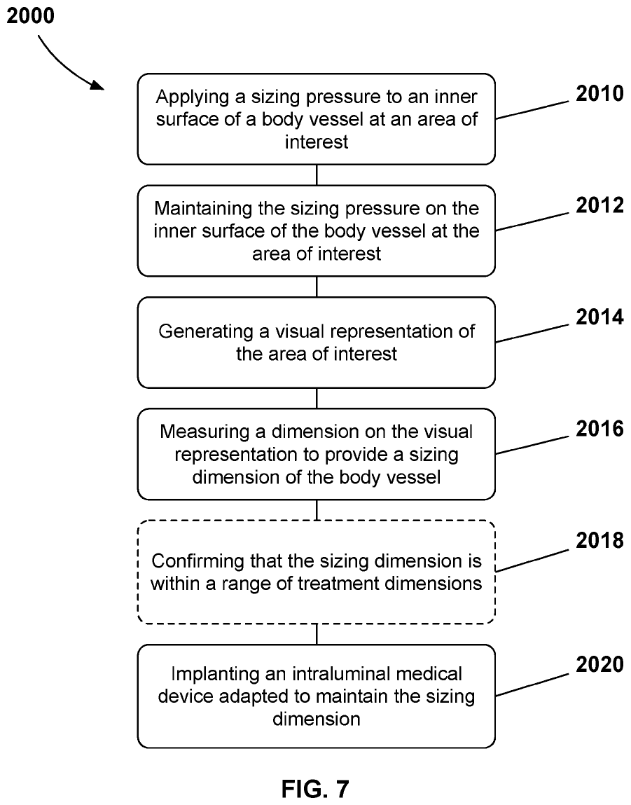

FIG. 8 illustrates another example method 2100 of implanting an intraluminal medical device at an area of interest in a body vessel. The method 2100 is similar to the method 1100 illustrated in FIG. 5, with the addition of steps 2020, 2022 required for implanting an intraluminal medical device. Accordingly, an initial step 2110 comprises positioning a pressure-exerting device within a body vessel at an area of interest. Another step 2112 comprises using the pressure-exerting device to apply a sizing pressure to an inner surface of the body vessel at the area of interest in the body vessel. Another step 2114 comprises maintaining the sizing pressure on the inner surface of the body vessel at the area of interest. Another step 2116 comprises generating a visual representation of the area of interest depicting a portion of the pressure-exerting device within the area of interest of the body vessel. Another step 2118 comprises measuring a dimension on the visual representation to provide the sizing dimension of the body vessel at the area of interest in the body vessel. Another step 2120, which is optional, comprises confirming that the sizing dimension of the body vessel provided by step 2118 is within a range of treatment dimensions of an intraluminal medical device. Another step 2122 comprises implanting an intraluminal medical device adapted to maintain the sizing dimension on the inner surface of a body vessel.

Advantageously, step 2116 of generating a visual representation of the area of interest is performed while the step 2114 of maintaining the sizing pressure on the surface of the body vessel at the area of interest is performed. This temporal relationship of the performance of these steps 2114, 2116 assures that the step 2118 of measuring a dimension on the visual representation provides the sizing dimension of the body vessel at the area of interest while the sizing pressure is maintained on the inner surface of the body vessel at the area of interest.

As with step 2020 in method 2000 illustrated in FIG. 8, any suitable intraluminal medical device and technique, including any desirable or necessary ancillary device, can be used in the performance of step 2122.

FIG. 9 illustrates another example method 2200 of implanting an intraluminal medical device at an area of interest in a body vessel. The method 2200 is similar to the method 1200 illustrated in FIG. 6, with the addition of steps 2220, 2222 required for implanting an intraluminal medical device. Accordingly, an initial step 2210 comprises navigating a sizing catheter having a compliant balloon through a body vessel to an area of interest in the body vessel. Another step 2212 comprises inflating the compliant balloon to an pressure of interest to apply a corresponding sizing pressure to an inner surface of the body vessel at the area of interest. Another step 2214 comprises maintaining the pressure of interest on the compliant balloon. Another step 2216 comprises generating a visual representation of the area of interest depicting the inflated compliant balloon within the body vessel at the area of interest. Another step 2218 comprises measuring a dimension of the inflated compliant balloon on the visual representation to provide a sizing dimension of the body vessel at the area of interest in the body vessel. Another step 2220, which is optional, comprises confirming that the sizing dimension of the body vessel provided by step 2218 is within a range of treatment dimensions of an intraluminal medical device. Another step 2222 comprises implanting an intraluminal medical device adapted to maintain the sizing dimension on the inner surface of a body vessel.

Advantageously, step 2216 of generating a visual representation of the area of interest is performed while the step 2214 of maintaining the sizing pressure on the surface of the body vessel at the area of interest is performed. This temporal relationship of the performance of these steps 2214, 2216 assures that the step 2218 of measuring a dimension on the visual representation provides a dimension that is the sizing dimension of the body vessel at the area of interest while the sizing pressure is maintained on the inner surface of the body vessel at the area of interest.

As with step 2120 in method 2100 illustrated in FIG. 8, any suitable intraluminal medical device and technique, including any desirable or necessary ancillary device, can be used in the performance of step 2222.

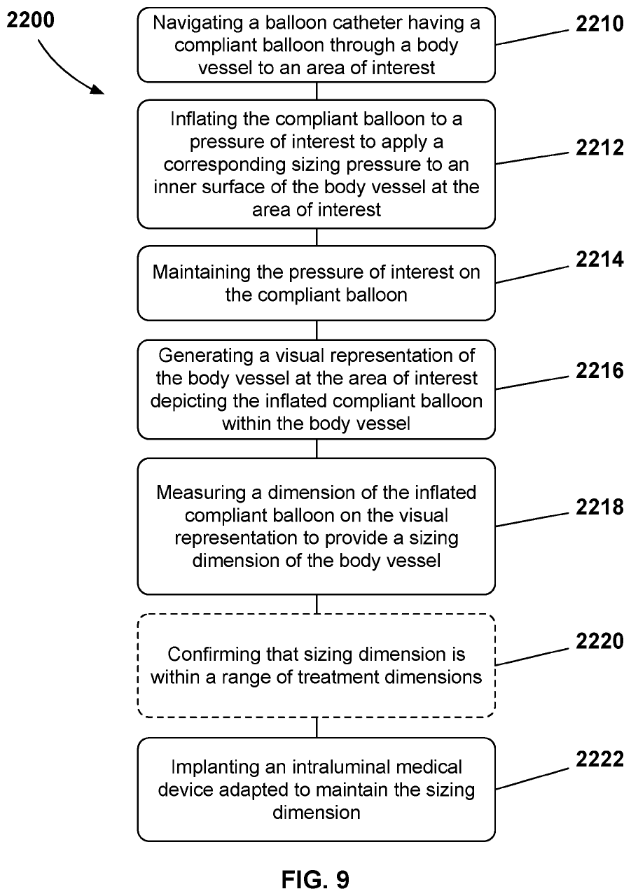

FIG. 10 illustrates another example method 3000 of implanting an intraluminal medical device at an area of interest in a body vessel. This method includes multiple steps of measuring a dimension on a visual representation of an area of an interest of a body vessel, each of which is performed while a different sizing pressure is maintained on the inner surface of the body vessel at the area of interest. The inclusion of these multiple measuring steps enables the generation of a curve of sizing pressures and corresponding measured dimensions, which is used in the selection of a desired dimension that, in turn, is used in the identifying of a corresponding pressure. Ultimately, an intraluminal medical device that is adapted to maintain the desired dimension of the inner surface of a body vessel is implanted at the area of interest in the body vessel.

The method 3000 is similar to the method 2000 illustrated in FIG. 7, with additional steps. Accordingly, an initial step 3010 comprises applying a first sizing pressure to an inner surface of a body vessel at an area of interest in the body vessel. Another step 3012 comprises maintaining the sizing pressure on the inner surface of the body vessel at the area of interest. Another step 3014 comprises generating a visual representation of the area of interest. Another step 3016 comprises measuring a dimension of the body vessel at the area of interest on the visual representation to provide a sizing dimension of the body vessel at the area of interest in the body vessel. Another step 3018 comprises repeating steps 3010, 3012, 3014, and 3016 using a second sizing pressure that is different from the first sizing pressure. Another step 3020 comprises generating a curve using the first and second pressures of interest and the corresponding first and second sizing dimensions. Another step 3022 comprises selecting a desired dimension on the curve. Another step 3024 comprises identifying the pressure that corresponds to the desired dimension on the curve. Another step 3026 comprises implanting an intraluminal medical device adapted to maintain the desired dimension on the inner surface of a body vessel at the area of interest in the body vessel. The cycle of steps 3010, 3012, 3014, and 3016, can be repeated in step 3018 any suitable number of times, including three times, four times, five times, six times, seven times, eight times, nine times, ten times, eleven times, twelve times, and more than twelve times. In each cycle of steps 3010, 3012, 3014, and 3016, the same or different sizing pressure from a prior cycle should be used, so long as at least two different pressures of interest are used across all cycles of steps 3010, 3012, 3014, and 3016. Advantageously, a different sizing pressure is used in each cycle of steps 3010, 3012, 3014, and 3016.

In some examples, step 3018, the repeating of steps 3010, 3012, 3014, and 3016, is performed using a constant increase in the sizing pressure across all steps 3010 of applying a sizing pressure performed in the performance of step 3018. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is less than about 20% of the dimension of the body vessel measured in step 3016 in the immediate prior cycle. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is less than about 10% of the dimension of the body vessel measured in step 3016 in the immediate prior cycle. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is less than about 5% of the dimension of the body vessel measured in step 3016 in the immediate prior cycle. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is less than about 2% of the dimension of the body vessel measured in step 3016 in the immediate prior cycle. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is less than about 2% but equal to or greater than about 1% of the dimension of the body vessel measured in step 3016 in the immediate prior cycle. In some examples, step 3018 is performed until such a constant increase in the sizing pressure applied in step 3010 in one cycle, as compared to the sizing pressure applied in step 3010 in the immediate prior cycle, results in an increase in the dimension of the body vessel measured in step 3016 of the one cycle as compared to the dimension of the body vessel measured in step 3016 in the immediate prior cycle, that is equal to or less than about 1% of the dimension of the body vessel measured in step 3016 in the immediate prior cycle.

Advantageously, for each cycle of steps 3010, 3012, 3014, and 3016, step 3014 of generating a visual representation of the area of interest is performed while the step 3012 of maintaining the sizing pressure on the surface of the body vessel at the area of interest is performed. This temporal relationship of the performance of these steps 3012, 3014 assures that the step 3016 of measuring a dimension on the visual representation provides a dimension that is the sizing dimension of the body vessel at the area of interest while the sizing pressure is maintained on the inner surface of the body vessel at the area of interest.

As with step 2020 in method 2000 illustrated in FIG. 7, any suitable intraluminal medical device and technique, including any desirable or necessary ancillary device, can be used in the performance of step 3026.

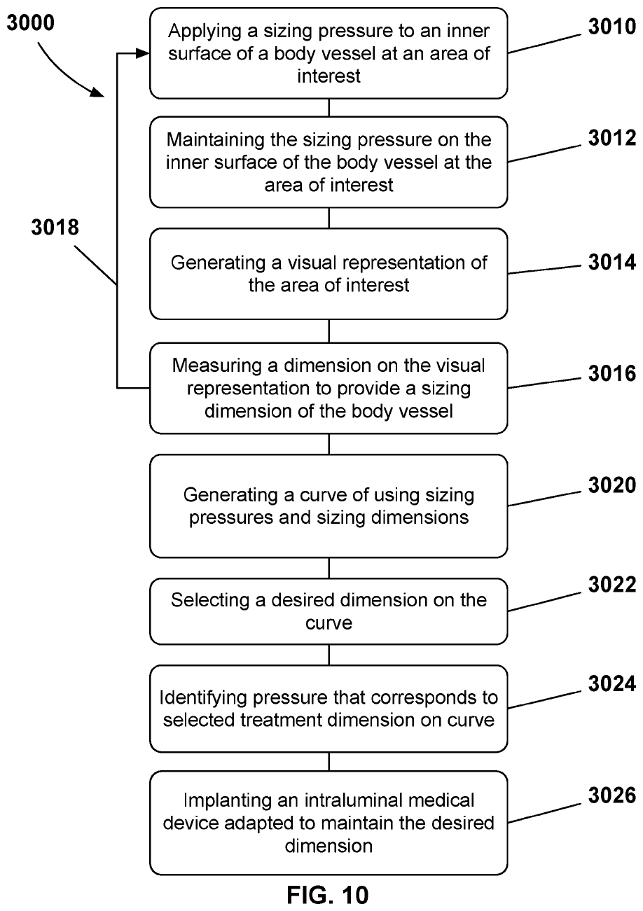

FIG. 11 illustrates an example method 4000 of making an intraluminal medical device. An initial step 4010 comprises applying a sizing pressure to an inner surface of a body vessel at an area of interest in the body vessel. Another step 4012 comprises maintaining the sizing pressure on the inner surface of the body vessel at the area of interest. Another step 4014 comprises generating a visual representation of the area of interest. Another step 4016 comprises measuring a dimension of the body vessel at the area of interest on the visual representation to provide a sizing dimension of the body vessel at the area of interest in the body vessel. Another step 4018 comprises making an intraluminal medical device adapted to maintain the sizing dimension of the inner surface of a body vessel at the area of interest in the body vessel.

Advantageously, step 4014 of generating a visual representation of the area of interest is performed while the step 4012 of maintaining the sizing pressure on the surface of the body vessel at the area of interest is performed. This temporal relationship of the performance of these steps 4012, 4014 assures that the step 4016 of measuring a dimension on the visual representation provides the sizing dimension of the body vessel at the area of interest while the sizing pressure is maintained on the inner surface of the body vessel at the area of interest.

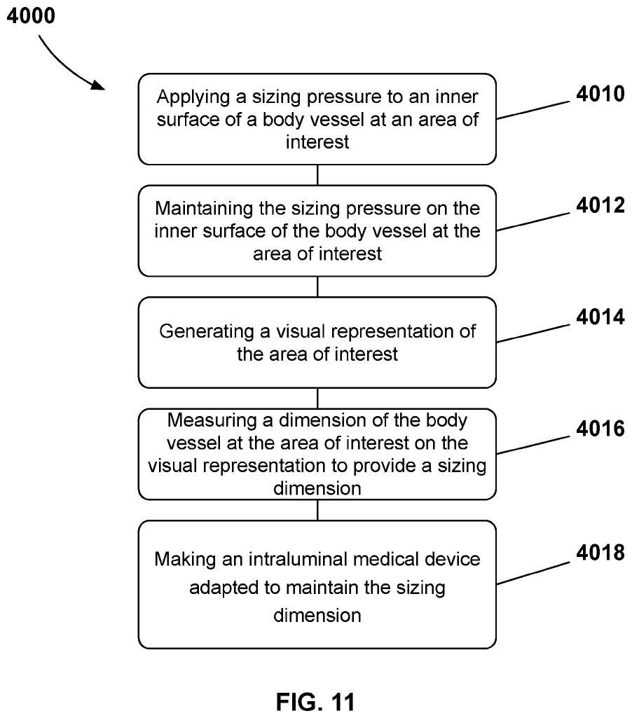

FIG. 12 illustrates an example method 5000 of making a plurality of intraluminal medical devices. An initial step 5010 comprises applying a first sizing pressure to an inner surface of a body vessel at an area of interest in the body vessel. Another step 5012 comprises maintaining the first sizing pressure on the inner surface of the body vessel at the area of interest. Another step 5014 comprises generating a visual representation of the area of interest. Another step 5016 comprises measuring a dimension of the body vessel at the area of interest on the visual representation to provide a sizing dimension of the body vessel at the area of interest in the body vessel. Another step 5018 comprises repeating steps 5010, 5012, 5014, and 5016 using a second sizing pressure that is different from the first sizing pressure. Another step 5020 comprises generating a curve using the first and second sizing pressures and the corresponding first and second sizing dimensions. Another step 5022 comprises making a first intraluminal medical device adapted to maintain a first dimension on the curve in the body vessel at the area of interest. Another step 5024 comprises making a second intraluminal medical device adapted to maintain a second dimension on the curve in the body vessel at the area of interest.

The cycle of steps 5010, 5012, 5014, and 5016, can be repeated in step 5018 any suitable number of times, including three times, four times, five times, six times, seven times, eight times, nine times, ten times, eleven times, twelve times, and more than twelve times. In each cycle of steps 5010, 5012, 5014, and 5016, the same or different sizing pressure from a prior cycle should be used, so long as at least two different pressures of interest are used across all cycles of steps 5010, 5012, 5014, and 5016. Advantageously, a different sizing pressure is used in each cycle of steps 5010, 5012, 5014, and 5016.

Advantageously, for each cycle of steps 5010, 5012, 5014, and 5016, step 5014 of generating a visual representation of the area of interest is performed while the step 5012 of maintaining the sizing pressure on the surface of the body vessel at the area of interest is performed. This temporal relationship of the performance of these steps 5012, 5014 assures that the step 5016 of measuring a dimension on the visual representation provides a dimension that is a sizing dimension of the body vessel at the area of interest while the inflation pressure of the sizing catheter is maintained on the inner surface of the body vessel at the area of interest.

### EXAMPLES

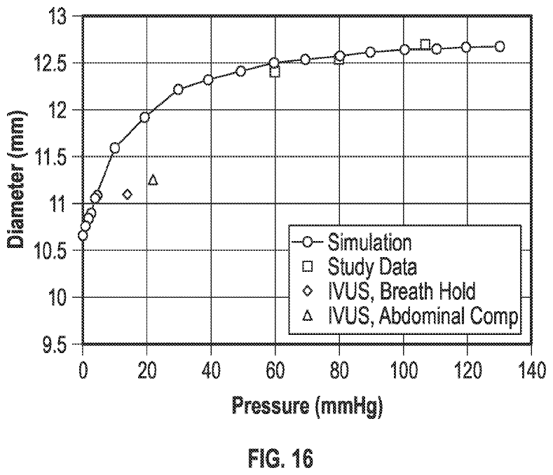

FIG. 16 illustrates a simulated pressure-diameter curve (“Simulation”), a pressure-diameter curve based on experimental data (“Study Data”) obtained using methods described herein, and pressure-diameter curves based on experimental data obtained using IVUS (“IVUS, Breath Hold” and “IVUS, Abdominal Comp”).

Table I, below, provides vessel sizing measurements obtained using each of the listed sizing methods in two cases. The measurements demonstrate the relative variability in sizing measurements obtained using conventional sizing methods (IVUS, Standing Duplex Ultrasound), and IVUS with Valsalve) as compared to the consistent sizing measurements obtained using methods described herein (sizing catheter at set pressure).

TABLE I
Vessel Sizing Methods and Measurements

| Sizing Method | Case 1 | Case 2 |
|---|---|---|
| IVUS | 6.9 mm | 10.2 mm |
| Standing Duplex US | 7.7 mm | 10.4 mm |
| IVUS w/Valsalva | 9.6 mm | 11.3 mm |
| Sizing Catheter at set pressure | 11.7 mm | 11.6 mm |

Those with ordinary skill in the art will appreciate that various modifications and alternatives for the described and illustrated examples can be developed in light of the overall teachings of the disclosure, and that the various elements and features of one example described and illustrated herein can be combined with various elements and features of another example without departing from the scope of the invention. Accordingly, the particular examples disclosed herein have been selected by the inventors simply to describe and illustrate examples of the invention and are not intended to limit the scope of the invention or its protection, which is to be given the full breadth of the appended claims and any and all equivalents thereof, including all claims made in all procedurally related applications.

What is claimed is:

1. A method of determining a sizing dimension of a body vessel at an area of interest in the body vessel, comprising:
navigating a sizing catheter having a compliant balloon through a body vessel to the area of interest in the body vessel;
inflating the compliant balloon to a pressure of interest to apply a corresponding sizing pressure to an inner surface of the body vessel at the area of interest;
maintaining the pressure of interest on the compliant balloon;
generating a visual representation of the area of interest depicting the inflated compliant balloon within the body vessel at the area of interest; and
measuring a dimension of the inflated compliant balloon on the visual representation to provide a sizing dimension of the body vessel at the area of interest in the body vessel.

2. The method of claim 1, wherein generating a visual representation of the area of interest is performed while maintaining the sizing pressure on the surface of the body vessel at the area of interest is performed.

3. The method of claim 1, wherein generating a visual representation of the area of interest is performed using one of radiography, X-ray computed tomography, fluoroscopy, venography, ultrasound, and magnetic resonance imaging.

4. The method of claim 1, wherein the visual representation comprises one of an image recorded on film and a digital still image.

5. The method of claim 1, wherein the visual representation comprises a series of digital still images.

6. The method of claim 1, wherein the visual representation comprises a digital video.

7. The method of claim 1, wherein the body vessel comprises a vein; and
wherein generating a visual representation of the area of interest includes injecting contrast into the balloon.

8. A method of implanting an intraluminal medical device at an area of interest in a body vessel, comprising:
applying a first sizing pressure to an inner surface of a body vessel at an area of interest in the body vessel;
maintaining the first sizing pressure on the inner surface of the body vessel at the area of interest;
generating a first visual representation of the area of interest;
measuring a first dimension of the body vessel at the area of interest on the visual representation to provide a first measured dimension of the body vessel at the area of interest in the body vessel;
applying a second sizing pressure to an inner surface of a body vessel at the area of interest in the body vessel, the second sizing pressure being different from the first sizing pressure
maintaining the second sizing pressure on the inner surface of the body vessel at the area of interest;
generating a second visual representation of the area of interest;
measuring a second dimension of the body vessel at the area of interest on the second visual representation to provide a second measured dimension of the body vessel at the area of interest in the body vessel;
generating a curve using the first and second sizing pressures and the corresponding first and second measured dimensions;
selecting a sizing dimension on the curve; and
implanting an intraluminal medical device adapted to maintain the sizing dimension within the body vessel at the area of interest in the body vessel.

9. The method of claim 8, wherein each of the steps of generating a visual representation of the area of interest is performed while a corresponding step of maintaining the sizing pressure on the surface of the body vessel at the area of interest is performed.

10. The method of claim 8, wherein each of the steps of applying a first sizing pressure to an inner surface of a body vessel and applying a second sizing pressure to an inner surface of a body vessel are performed with a sizing catheter having a compliant balloon.

11. The method of claim 10, wherein the body vessel comprises a vein; and
wherein each of generating a first visual representation of the area of interest and generating a second visual representation of the area of interest includes injecting contrast into the balloon.

12. The method of claim 8, wherein each of the steps of generating a first visual representation of the area of interest and generating a second visual representation of the area of interest is performed using one of radiography, X-ray computed tomography, fluoroscopy, venography, ultrasound, and magnetic resonance imaging.

13. The method of claim 8, wherein each of the first visual representation and the second visual representation comprises one of an image recorded on film and a digital still image.

14. The method of claim 8, wherein each of the first visual representation and the second visual representation comprises a series of digital still images.

15. The method of claim 8, wherein each of the first visual representation and the second visual representation comprises a digital video.

16. A method of making a plurality of intraluminal medical devices, comprising:
applying a first sizing pressure to an inner surface of a body vessel at an area of interest in the body vessel;
maintaining the first sizing pressure on the inner surface of the body vessel at the area of interest;
generating a first visual representation of the area of interest;
measuring a first dimension of the body vessel at the area of interest on the visual representation to provide a first measured dimension of the body vessel at the area of interest in the body vessel;
applying a second sizing pressure to an inner surface of a body vessel at the area of interest in the body vessel, the second sizing pressure being different from the first sizing pressure
maintaining the second sizing pressure on the inner surface of the body vessel at the area of interest;
generating a second visual representation of the area of interest;
measuring a second dimension of the body vessel at the area of interest on the second visual representation to provide a second measured dimension of the body vessel at the area of interest in the body vessel;
generating a curve using the first and second sizing pressures and the corresponding first and second measured dimensions;
selecting a first pressure of interest on the curve;
selecting a second pressure of interest on the curve;
making a first intraluminal medical device adapted to exert the first pressure of interest on an inner surface of the body vessel; and
making a second intraluminal medical device adapted to exert the second pressure of interest on an inner surface of the body vessel.

17. The method of claim 16, wherein the step of generating a first visual representation of the area of interest is performed while the step of maintaining the first sizing pressure on the inner surface of the body vessel at the area of interest is performed; and
wherein the step of generating a second visual representation of the area of interest is performed while the step of maintaining the second sizing pressure on the inner surface of the body vessel at the area of interest is performed.

18. The method of claim 17, wherein each of the steps of applying a first sizing pressure to an inner surface of a body vessel and applying a second sizing pressure to an inner surface of a body vessel are performed with a sizing catheter having a compliant balloon.

19. The method of claim 18, wherein the body vessel comprises a vein; and
wherein each of generating a first visual representation of the area of interest and generating a second visual representation of the area of interest includes injecting contrast into the balloon.

20. The method of claim 16, wherein each of the steps of generating a first visual representation of the area of interest and generating a second visual representation of the area of interest is performed using one of radiography, X-ray computed tomography, fluoroscopy, venography, ultrasound, and magnetic resonance imaging.
